(19) United States
(12) Patent Application Publication
(10) Pub. No.: US 2007/0134195 A1
(43) Pub. Date: Jun. 14, 2007

(54) Topical Analgesic for Sensitive Skin

(76) Inventors: Aurelia L. Ward, Middletown, DE (US); Kennon B. Ward, Middletown, DE (US)

Correspondence Address: WHITE-WELKER & WELKER, LLC, P.O. BOX 199, CLEAR SPRING, MD, 21722-0199, US

(21) Appl. No.: 11/609,330

(22) Filed: Dec. 11, 2006

Related U.S. Application Data
(60) Provisional application No. 60/750,091, filed on Dec. 13, 2005.

(51) Int. Cl. A61K 36/886; A61K 36/53; A61K 36/328; A61K 36/28

(52) U.S. Cl. 424/74; 424/748; 424/769; 424/764; 424/771; 424/745; 424/744

(57) ABSTRACT

A topical analgesic for sensitive skin for providing a fast-acting and deeply penetrating topical analgesic that does not irritate sensitive skin teaching a chemical composition consisting of one or more herbs containing the properties of an analgesic such as those selected from a group of herbs containing methyl salicylate, salicin, or menthol, including birch bark, wintergreen, willow bark meadowsweet and peppermint or any other herbs with analgesic properties, such as but not limited to, lavender, Arnica Montana, passion flower, calendula, chamomile; a transdermal agent, such as Arnica Montana; one or more herbs containing the properties of an anti-inflammatory, such as lavender or birch bark; Myrrh; and a base to hold the ingredients together and make it applicable to skin. The topical analgesic may also include a natural preservative to increase product stability and shelf life and one or more essential oils for added pain relief using olfactory senses.

### CROSS REFERENCE TO RELATED APPLICATIONS

This application claims priority from U.S. Provisional Patent Application Ser. No. 60/750,091, entitled “Topical Analgesic for Sensitive Skin”, filed on Dec. 13, 2005.

### SEQUENCE LISTING OR PROGRAM

Not Applicable

### FEDERALLY SPONSORED RESEARCH

Not Applicable

### TECHNICAL FIELD OF THE INVENTION

The present invention relates generally to pain relieving cream. More specifically, the present invention relates to a topical analgesic for sensitive skin that provides a fast acting and deeply penetrating topical analgesic, which does not irritate sensitive skin.

### BACKGROUND OF THE INVENTION

It can be appreciated that pain-relieving cream has been in use for years. Presently, topical analgesics typically found on the market include creams and sprays such as Aspercreme, Icy Hot, Fluid Joint, Stopain spray, and Blue Emu Ice Spray.

The main problem with conventional pain relieving cream is that almost all use capsaicin, camphor, methyl salicylate and/or menthol, which carry a potent, medicinal, and unpleasant scent. Another problem with conventional pain relieving cream is a significant percentage of the general population is sensitive to these remedies, and develops contact dermatitis, burning sensations, and/or other discomforts. In addition, none of the products presently on the market can safely be used over broken skin.

Another problem with conventional pain relieving cream is topical analgesics presently on the market penetrate only superficially into the skin, addressing only the pain in the outer layers of the muscle. The resulting partial pain relief is short-lived, needing re-application within a few hours.

U.S. Pat. No. 6,579,543 entitled “Composition for topical application to skin” issued to McClung on Jun. 17, 2003 teaches a composition for topical application to an animal's skin for relief from a variety of symptoms caused by medical conditions or physical injuries. The composition includes at least one composition having analgesic activity, at least one composition having anti-inflammatory activity, at least one composition having antioxidant activity, at least one composition having anti-neuralgic activity, at least one composition having blood circulation promotion activity, and at least one composition having antidepressant activity. A method for relieving pain by topical application of the composition is also provided.

McClung teaches a composition which includes groupings of herbs in 6 groups: analgesic, anti-inflammatory, anti-oxidant, anti-neuralgic, blood circulation, and anti-depressant-along with preservative, and base, but excludes myrrh.

The present invention improves upon McClung and is distinguished by its use of Myrrh and other essential ingredients in the compound that not only includes myrrh but a transdermal agent such as Arnica Montana, an herbal analgesic, an herbal anti-inflammatory, a skin conditioner for healing cuts and bruises, a natural preservative if necessary or desired, and a base for application to skin. The present invention teaches that myrrh as a first essential oil combined with lavender, a second essential oil, and/or birch bark, a third essential oil, and/or calendula oil to make an effective pain reliever. Unexpected results show that the myrrh acts stronger with another analgesic than each individually.

U.S. Pat. No. 6,528,076 entitled “Topical compositions and methods for treating pain” issued to Small on Mar. 4, 2003 teaches topical compositions and methods for using the same to relieve pain in humans, such as pain associated with cancer, bone injuries, old age, and many other forms of pain. Specifically, the present compositions comprise an effective amount of acetone, a salicylate-based composition and an emollient. In addition, the present compositions further comprise one or more compositions including, but not limited to, terpenes and essential oils. The present compositions may be in the form of a liquid, preferably a slightly viscous liquid, or other forms including, but not limited to, salves, creams, gels, ointments or sprays.

U.S. Pat. No. 4,521,411 entitled “Analgesic composition” issued to Koloff on Jun. 4, 1985 teaches an externally applicable analgesic composition comprises an extract derived from sumac leaves, sassafras root, oak tree bark and an alcohol component, combined with benzocaine, procaine and menthol components. The topical application of this composition has proven effective for the temporary relief of pain and stiffness associated with arthritis, bursitis, muscle cramp and other aches and pains.

U.S. Pat. No. 5,223,257 entitled “Topical composition for relieving aches and pains” issued to Arora on Jun. 29, 1993 teaches a composition comprising approximately equal proportions by volume of wintergreen oil, olive oil and/or oil of Eucalyptus and alcohol has an analgesic effect in treating aches and pains when rubbed or massaged on skin covering tissue from which such aches or pains emanate.

While these devices may be suitable for the particular purpose to which they address, they are not as suitable for providing a fast-acting and deeply penetrating topical analgesic that does not irritate those with sensitive skin. The main problem with conventional pain relieving cream is almost all are based on an older technology that uses capsaicin, camphor, methyl salicylate and/or menthol, which carries a potent, medicinal and unpleasant scent.

Another problem is the significant percentage of the general population is sensitive to these remedies, which results in contact dermatitis, burning sensations, and/or other discomforts. None of the products known in the prior art can safely be used over broken skin. Also, another problem is topical analgesics presently on the market penetrate only superficially into the skin, addressing only the pain in the outer layers of the muscle. The resulting partial pain relief is short-lived, needing re-application within a few hours.

### SUMMARY OF THE INVENTION

In view of the foregoing disadvantages inherent in the known types of pain relieving cream now present in the prior art, the present invention provides a new topical analgesic for sensitive skin construction wherein the same can be utilized for providing a fast-acting and deeply penetrating topical analgesic that does not irritate those with sensitive skin.

The general purpose of the present invention is to provide a new topical analgesic for sensitive skin that has the advantages of the pain relieving cream mentioned previously and many novel features that result in a new topical analgesic for sensitive skin which is not anticipated, rendered obvious, suggested, or even implied by any of the prior art pain relieving cream, either alone or in any combination thereof.

The present invention generally is an all vegetarian chemical mixture which comprises the following compositions: essential oil of Myrrh, Birch Bark essential oil, Arnica Oil, Wintergreen essential oil, and willow bark in a cream base of equal parts shea and aloe butter. The mixture of the present invention, in a preferred embodiment, is made of the following compositions and ratios: 2-10% Myrrh essential oil, 20% solution of Arnica oil in a carrier oil, approx. 4%, and 2-4% Birch Bark essential oil.

A primary objective of the present invention is to provide a topical analgesic for sensitive skin that will overcome the shortcomings of the prior art devices while providing a fast-acting and deeply penetrating topical analgesic that does not irritate those with sensitive skin.

Another object is to provide a topical analgesic for sensitive skin that does not use alcohol as a base, which dries the skin.

Another object is to provide a topical analgesic for sensitive skin that relieves muscle pain on contact, as well as quick, penetrating relief on tendons and ligaments.

Another object is to provide a topical analgesic for sensitive skin that does not cause contact dermatitis to the general population, is safer to use on sensitive skin, and does not convey a strong unpleasant scent.

Another object is to provide a topical analgesic for sensitive skin that can be used over broken skin, and minor scrapes that heals the outer layers of skin as well as the deepest layers of muscles.

Another object is to provide a topical analgesic for sensitive skin that has vegetarian ingredients.

The present invention is novel in that it incorporates two methods of pain relief in a topical composition: herbs entering the skin for pain relief directly on site as well as using olfactory senses to directly stimulate the brain. Thus it is an objective of the present invention to teach a composition that includes delivery of pain relief directly to the body through the direct contact with the skin in addition to pain relief additionally delivered via airborne scent to the brain.

An additional level of pain relief that is employed by the present invention, which has recently been proven by Australian researchers, Drs. John Prescott and Jenelle Wilkie, is that sweet smelling odors, which affect our mood, also do have an analgesic effect. Pain, which is a subjective experience, is controlled largely by our brains, thoughts, and feelings. The olfactory senses are the only direct path to the brain, entering the brain through the limbic system, which affects emotions, memories, as well as release neurotransmitters including endorphins, which reduce pain, serotonin, and promotes relaxation.

In these respects, the topical analgesic for sensitive skin according to the present invention substantially departs from the conventional concepts and designs of the prior art, and in so doing provides an apparatus primarily developed for the purpose of providing a fast-acting and deeply penetrating topical analgesic that does not irritate those with sensitive skin.

### BRIEF DESCRIPTION OF THE DRAWINGS

Not Applicable

### DETAILED DESCRIPTION AND BEST MODE OF THE INVENTION

In the following detailed description of the invention a plurality of exemplary embodiments of the invention are taught. These embodiments are described in sufficient detail to enable those skilled in the art to practice the invention, but other embodiments may be utilized and logical, mechanical, electrical, chemical, and other changes may be made without departing from the scope of the present invention. The following detailed description is therefore, not to be taken in a limiting sense, and the scope of the present invention is defined only by the appended claims.

In the following description, numerous specific details are set forth to provide a thorough understanding of the invention. However, it is understood that the invention may be practiced without these specific details. In other instances, well-known structures and techniques known to one of ordinary skill in the art have not been shown in detail in order not to obscure the invention.

Composition

The present invention generally is a chemical mixture, that is also vegetarian in nature, which comprises the following compositions: essential oil of Myrrh, Birch Bark essential oil, Arnica Oil, Wintergreen essential oil, and willow bark in a cream base of equal parts shea and aloe butter. The mixture of the present invention, in a preferred embodiment, is made of the following compositions and ratios: 2-10% Myrrh essential oil, 20% solution of Arnica oil in a carrier oil, approx. 4%, and 2-4% Birch Bark essential oil.

Myrrh is a powerful antiseptic resin useful on skin sores, wounds, and cuts. It also is an anti-inflammatory, antibacterial, anti-fungal, astringent, and expectorant with analgesic properties. Myrrh has been used for abrasions, gingivitis, pulmonary congestion, and wounds, as well as in mouthwash to treat ulcers and even in toothpaste. Myrrh activates blood circulation, reduces swelling, and aids regeneration of damaged tissue. Myrrh is primarily used as a flavoring agent and as a fragrance. When used externally in large doses, myrrh can cause dermatitis. It is not recommended taken internally for pregnant or breastfeeding women. However, in the present invention, myrrh is used externally in small enough doses as not to potentially irritate the skin while maintaining its effectiveness.

In the mixture of the present invention, it is used for its superior analgesic and anti-inflammatory effects in essential oil form, as well as for its antiseptic and healing properties. Myrrh can be found in its resin form, as an essential oil, and in capsules, mouthwashes, tinctures, extracts, and salves.

Arnica Montana is an herb that grows wild in many parts of Europe. It is an anti-inflammatory and an antibacterial agent in vitro. Arnica is claimed to be useful for relieving muscle and joint aches and is frequently cited in herbal literature as being able to promote wound healing. Arnica has also been found to act as an excellent transdermal agent, which is its primary use in this mixture. Arnica's active ingredients are thought to be flavonoid glycosides and sesquiterpenoid lactones. Arnica can be found in use in the form of spray, gel, cream ointment, tablets, teas, and tinctures. In the mixture of the present invention a 20% solution of Arnica oil in carrier oil totaling approximately 4% of the chemical composition is specified. The concentration of Arnica is kept to a minimum since it is a known skin irritant.

Other transdermal agents include flax seed oil, emu oil and ben seed oil. However there are numerous manmade transdermal agents that could be used or substituted in the compositions taught by the present invention but that have not explored in an effort to keep the composition comprised of 100% natural compounds.

Essential oil of birch bark contains methyl salicylate, which has analgesic, anti-inflammatory and antipyretic properties. It has been shown to exert diuretic properties. The benefits of birch bark include relief of headache and other analgesic effects as well as treatment of various acute and chronic skin disorders, GI disorders and kidney stones.

Essential oil of birch bark is claimed to act against bladder infections, gout, neuralgia, rheumatism, and tuberculous cervical lymphadenitis. In Veterinary medicine, essential oil of birch bark has been used to treat various skin disorders. Birch is used topically as a treatment for bruises, burns, eczema and wounds; as a bath herb for skin eruptions; and as a liniment for rheumatism. Birch bark is found in cut, shredded, or in oil form. In the mixture of the present invention the chemical composition is made of 2-4% Birch Bark essential oil.

Arnica oil is a transdermal agent that absorbs quickly and easily into the muscles, ligament, and tendons. The glycosides in amica oil bond to the myrrh and birch bark oils and are carried through the skin into the muscles via the sesquiterpenoid lactones in the arnica oil. The myrrh is an analgesic and anti-inflammatory giving quick relief to muscle aches, pulls, strains, or other muscle injury, as well as relief for joint aches. The birch bark contains methyl salicylate and acts as an anti-inflammatory and analgesic. Test have shown that increasing the myrrh concentration in the mixture from 2% to 10% to more than double the length of time of pain relief. Results indicate that myrrh, when paired with amica, a transdermal agent, is superior in pain relief over other popular topical pain relievers in the present industry in both speed and duration of relief without apparent negative side effects. The birch bark contains methyl salicylate and acts as an anti-inflammatory and analgesic.

TABLE 1

| | | | | Arnica |
|---|---|---|---|---|
| | Methyl | Salicin | Myrrh | Montana |
| | Salicylate | Concen- | Concen- | Concen- |
| Result | Concentration | tration | tration | tration |
| 4-6 Hours of Relief | 2% Birch Bark | 2% | 2% | .8-2% |
| (Base Mixture) | and | Willow | | |
| | 2% Wintergreen | | | |
| Alternative Mixture | 4% Birch Bark | 2% | 2% | .8-2% |
| | | Willow | | |
| 8-9 Hours of Relief | 6% Birch Bark | N/A | 2% | .8% or 2% |
| 10-12 Hours of | 4-6% Birch | N/A | 8% | .8% |
| Relief | Bark | | | |
| 16-24 Hours of | 2% Birch Bark | N/A | 10% | .8% |
| Relief | | | | |

Referring to Table 1, in a first mixture willow bark is used for its salicin content. This main ingredient, salicin, is also found in meadowsweet, which could be substituted. Birch Bark has a slightly higher concentration of methyl salicylate than wintergreen, while also offering fewer potential side effects and a reduced possibility of skin irritation when applied. Synthetic methyl salicylate can also be substituted for birch bark. In testing, when varying the concentration by increasing the percentage of birch bark by substituting in place of Salicin and Wintergreen, it was unexpectantly discovered that the mixture was more effective, but had a potent smell that would likely not be agreeable to a user.

Again referring to Table 1, the base mixture is comprised of a 2% mixture of birch bark essential oil and wintergreen essential oil, myrrh essential oil, birch bark essential oil, willow bark, and arnica montana. It is also important to note that when using a 20% mixture of arnica in an oil base, the percentage of actual arnica montana is reduced from 2% to 0.8%. The oil base of arnica montana is less than 4%.

Now referring to Table 2, experimental ranges and results are shown for various embodiments of the base mixture with varying concentrations of myrrh and birch. Experimental results have shown that by varying the concentration and ratio of myrrh and birch, the mixture of the present invention can be adjusted for an extended effective time or more pain relief after application.

TABLE 2

| Goal | Concentrations | Results |
|---|---|---|
| 8-9 Hours of Relief | 2% Myrrh with | More Effective than Base |
| | 6% Birch | Mixture, Immediate Relief |
| 10 Hours or More of | 8% Myrrh with | More Effective than 2% |
| Relief | 6% Birch | Myrrh |
| 10 Hours or More of | 8% Myrrh with | No Noticeable Difference |
| Relief | 4% Birch | from 6% Birch |
| Resulting Pain Relief | 0% Myrrh with | Less Effective than any |
| only about 2 Hours | 8% Birch | Other Mixture. Pain Relief |
| | | ONLY after 30 Minutes |
| | | of Application |
| 16-24 Hours or More | 10% Myrrh with | Most Effective Mixture, |
| of Relief | 2% Birch | Immediate Pain Relief |
| | | and Longest Lasting |

This composition of the present invention can also be comprised substituting wintergreen and/or willow bark in place of ⅓ or ⅔ of the birch bark for the same effect, or any other combination of herbs containing salicin, such as meadowsweet or methyl salicylate.

The composition of the present invention can also be comprised by substituting synthetic methyl salicylate in place of essential oil of birch bark, wintergreen, and/or willow bark. This substitution will increase the benefits to sensitive skin.

In an alternative form, Grape seed oil or other oils such as jojoba can be used as a base to form a liquid for a roll-on. Grape seed oil is also a transdermal agent acting with the arnica producing a quick absorbing non-greasy mixture. For a longer shelf life, fractionated coconut oil may also be used, as this oil has an indefinite shelf life, and is also light with a non-greasy mixture, and incorporates the essential oils well for ease into the skin. Varying the base can also produce a spray, gel or bath soak.

In place of using essential oils, a ratio of one-ounce raw herbs to six ounces base heated for approximately thirteen hours at 110-120 degrees Fahrenheit. This will render a similar effect as the uses of essential oils; the main difference being the time it takes to render a final product. Using essential oils, there is no heating outside of melting of the shealoe butter used as the base at the same heat range.

Arnica oil is a transdermal agent that is absorbed into the muscles, ligament, and tendons. The glycosides in arnica oil bond to the myrrh and birch bark oils and are carried through the skin into the muscles via the sesquiterpenoid lactones in the arnica oil. The myrrh is an analgesic and anti-inflammatory giving quick relief to muscle aches, pulls strains, or other muscle injury, as well as relief for joint aches. The birch bark contains methyl salicylate and acts as an anti-inflammatory and analgesic.

Unexpected results from the composition show that it can be modified using the same or similar main ingredients previously taught with minor alterations such as the addition of skin soothing herbs such as chaulmoogra and/or calendula to produce a product line of topical pain-relieving products. The composition has been discovered to have effectiveness on not only sensitive skin but also those with skin irritations such as excema and certain types of psoriasis. Ongoing research is being conducted to discover the extent of this effectiveness. Additionally, it has recently been discovered that the composition is also effective on preventing scarring from minor scratches and cuts on those prone to scarring from such.

The composition has shown effectiveness as a moisturizer while soothing skin irritation as it takes away muscle aches as well as chronic neck and back pain, strains, pulls, knots, tension headache, and pain from fibromyalgia. It has even been reported effective on relieving pain and speeding the healing on sutured skin from surgery.

The groupings of main ingredients include (A) one or more herbs containing the properties of an analgesic. This could be in the form of herbs containing methyl salicylate, salicin, menthol, or other herbs with natural analgesic properties, such as lavender, everlast, or calendula, just to name a few. Methyl salicylate is not required as one of the analgesics; however, methyl salicylate and similar compositions occur naturally in herbs such as: birch bark, wintergreen, willow bark (salicin), meadowsweet (salicin), and peppermint (menthol); (B) a transdermal agent, such as Arnica Montana or emu oil; (C) herb(s) with the property of an anti-inflammatory, such as lavender or birch bark; (D) the addition of one or more herbs with the properties of healing cuts or wounds such as calendula and/or chaulmoogra; (E) Myrrh for its supporting properties and anti-microbial, anti-bacterial effects; and (F) a base to hold the ingredients together and make it applicable to skin. A natural preservative may or may not be added at this point for the purpose of increasing shelf life and stability.

Calendula oil is most recognized for its ability to help heal wounds and abrasions. The carrier oil is created by infusing calendula petals in soybean oil; 10 parts calendula oil is equal to 1-part blossoms.

Chaulmoogra oil is also known as Gynocardia oil or Hydnocarpus oil. This oil is believed to have antimicrobial properties and is used to treat eczema, psoriasis, arthritis, sprains, bruises, and skin inflammations. Studies have shown it to be effective treatment for early stages of leprosy. The oil is cold-pressed physically from the seeds of chaulmoogra to retain its properties.

Fractionated Coconut oil contains only the medium-chain triglycerides (MCTs) of coconut oil, making it a liquid and very stable oil. It is produced through hydrolysis of coconut oil and is then fractionated by steam distillation to isolate the MCTs. Fractionated coconut oil has indefinite shelf life and is a light, non-greasy, non-staining, liquid oil. It is a very good choice for use with essential oils, as it helps carry therapeutic oils under the skin.

Method of Production

The method for making the composition taught by the present invention is a multi-step process that includes herbal extraction, Essential Oils, and the combination of the herbal extraction with a minimal amount of essential oil as required to create the desired composition. Substituting essential oils for dried herb extraction makes the production process faster and easier, but also prone to separation if not kept at moderate to low temperatures.

In a first step the base is heated if using a solid such as a butter to a liquid state. In one embodiment, shealoe butter, a 50/50 premix of shea butter and aloe butter is used. The butter is melted at low temperature in a crock-pot. After the butter is melted, supporting carrier oils are added followed by the addition of any desired essential oils. Tables 3 and 4 illustrate the possible ranges of supporting carrier oils and essential oils that may be added and the desired percentage range in the composition. Other essential oils that contribute to pain relief may be added or substituted to vary scent and type of pain relief.

TABLE 3

| Supporting Carrier Oils | Concentrations |
|---|---|
| Calendula | 5-10% |
| Arnica | 4% |
| Chaulmoogra | 5-10% |
| Aloe Vera (if base does not already contain aloe) | 10% |
| Meadowfoam | 2-5% |

TABLE 4

| Essential Oils | Concentrations |
|---|---|
| Birch bark or wintergreen | 2%, and/or lavender 3-5% |
| Myrrh | 8-10% |
| Ginger | ½-1% |
| Allspice | ½% |
| Coriander | 1% |

The use of essential oils as the main ingredients has been found to be problematic for multiple reasons. They shorten the shelf life of the composition to approx. 6 months to 1 year without the use of preservatives. High quality essential oils are expensive and large quantities of high quality essential oils are hard to obtain consistently. And the oils need to be protected from heat and light to maintain its quality and potency and have a tendency to separate if not stored in a cool dark environment.

In an alternative method of production the combination of the herbal extraction with the minimum essential oil addition is combined. This is a longer process, but there are several benefits to using this mixed method. Materials are easier to obtain and less expensive and using herbal extraction allows for greater control of scent, and allows for a scent-free version. A greater variety of scent can be administered while staying all-natural through the use of essential oils, however in lesser quantity. The composition can sustain a much longer shelf life. And the cream base is more stable, depending on base used, and less prone to separation.

The shortcomings of the combination method are that it is more difficult to identify actual percentage of extracted herbs and resin and it is a longer process for extraction. The medium for extracting herbs must be able to sustain higher temperatures; if water is used, care must be taken to boil the water off less it become an avenue for bacteria to enter the composition. Finally, it is not completely proven that the myrrh resin will disallow the growth of bacteria, even as it is an anti-microbial.

Method Steps

First, the dried herbs must be tied off into bundles using cheesecloth and string. Some of the larger amounts of dried herb may need to be divided into smaller bundles. The final concentration after extraction and combination with the base cream or oil is illustrated in FIG. 5

TABLE 5

| Ingredient | Example I |
|---|---|
| Birch bark | 2-4% |
| Myrrh | 5%-10% depending if essential oil is added later |
| | with other essential oils |
| Lavender | 3-4% |
| Calendula | 3-4% |
| Arnica Montana | 1-2% |

Next, these herb bundles are placed in a crock pot where approx. 12-16 hours of low boiling in a base that is not a butter can occur. At the end of the extraction period, the liquid should be a dark brown), the bundles are further squeezed to remove excess liquid and removed from the pot. The liquid tea is allowed to cool before shealoe butter is added for the cream base. The shealoe is heated, careful not to overheat, as it would ruin consistency of the butter. Once the butter is completely dissolved within the mixture tea, it is stirred. At this point, the 6-10% chaulmoogra oil, 2-4% myrrh essential oil, and 1-3% spike lavender essential oil and/or another oil to modify scent is added.

The purpose behind adding essential oil at this time is twofold: it adds a pleasant but mild aroma to the composition, enhancing the soothing ability to the olfactory senses, and further enhances strength of the dried herbs.

In a specific example, 300 mL of Turkey Red oil is used as a beginning base. This oil was chosen for its ability to emulsify in water. 1450 mL of water was mixed with the oil to create enough liquid to cover the following cheesecloth bags of herbs: 21 g Arnica Montana, 40 g Birch Bark (Betula alba), 29 g Calendula, 33 g Lavender, and 45 mL myrrh resin.

The mixture was then cooked down on low heat over approx. 15 hours or more to cook out or evaporate the added water. The remaining liquid is then combined with the cream base, in this case, shealoe butter, at a ratio of approx 1:2. The butter is gradually melted over low heat until liquid and mixed well with the herbal tea. Once combined, the chaulmoogra oil is added, followed by the myrrh and lavender essential oils. The oils are added after the heat has been shut off.

The base in the composition, as well as amount of ingredients can be modified to produce variations for a plurality of topically applied pain-relieving products.

Bath-Soaking Cubes are all over body aches cubes to relieve pain after a hard day, as well as pain from fibromyalgia. The pleasant aroma cubes melt into the bath water for a soothing relaxing soak with the potential for an oil to be added to the bath for the same effect.

Massage Oil is a pain relieving composition for a soothing body massage that assists masseurs in relieving pain through the oils while they massage for a double effect.

After Workout Rejuvenating Body Spritz is an after workout product for the casual or hardcore workout person: this spray-on composition is a jumpstart on achy muscles before they even start.

Foot Spa is a soaking bath for the feet or a rub-on cream for tired achy feet: this product provides the need for relief after a long day that is beneficial for those with diabetes or neuropathy.

A Hiker's Blend with Insect Repellent is a pain relieving cream with essential oils added for insect repelling, such as Cedar.

An add-on for First Aid kits, which could be perceived to be a sibling to the Hiker's Blend with Insect Repellent; this pain reliever acts as a Neosporin and moisturizer/minor insect bite/sting reliever.

A Sinus relieving gel where the wintergreen smells from the birch bark is released. This simple and effective derivative is used to clear sinuses.

Scent Compositions

In an alternative embodiment scent varieties can be used to enhance the pain relieving properties and provide an aromatherapy element to the compositions previously taught. Lavender combinations such as: straight lavender, vanilla lavender, lavender and clary sage mix, lavender and myrrh mix for woodsy scent are suggested. Other scent possibilities that have been shown to support pain relief include essential oils of grapefruit and popular spicy essential oils such as cinnamon, sage, clove, allspice essential oils as well as any other scent that relieves endorphins in the brain.

The scent compounds could be different for different populations such as neroli, ylang ylang, long used in Eastern cultures but no studies are available in the prior art to teach or suggest which scents may be preferred on a geographical basis or have a direct correlation with healing. However, it is suspected that any scent, which causes the release of endorphins and results in pleasure to the brain, would be preferred and can be used.

Typically scents in the form of essential oils would generally be added in a 1-3% concentration. For stronger scents, such as clove and allspice, the added amount would be reduced to ½%.

In yet another alternative combinations for skin conditioning and skin conditions, the chaulmoogra and/or calendula would range in 5-10% concentrations. Other herbs that are gentle to skin and supporting of healing bruising and skin conditions such as chamomile could also be used. Again, lavender and evening primrose also falls into this category as well, to name only a few.

As to a further discussion of the manner of usage and operation of the present invention, the same should be apparent from the above description. Accordingly, no further discussion relating to the manner of usage and operation will be provided.

It is appreciated that the relationships for the parts of the invention, to include variations, are deemed readily apparent and obvious to one of ordinary skill in the art, and all equivalent relationships in the above description are intended to be encompassed by the present invention.

In addition, other areas of art may benefit from this method and adjustments to the design are anticipated. Thus, the scope of the invention should be determined by the appended claims and their legal equivalents, rather than by the examples given.

The embodiments of the invention in which an exclusive property or privilege is claimed are defined as follows:

1. A topical analgesic for sensitive skin, the chemical composition comprising:
from about 2 to about 10 weight percentage Myrrh essential oil,
from about 2 to about 4 weight percentage Birch Bark essential oil,
from about a 20% solution of Arnica Montana oil in a carrier oil totaling about 4% weight percentage of the chemical composition,
in from about 92% to about 82% weight percentage of a cream base of equal parts shea and aloe butter.

2. The topical analgesic for sensitive skin, the chemical composition of claim 1 further comprising:
about 2 weight percentage Birch Bark essential oil,
about 2 weight percentage Wintergreen essential oil,
about 2 weight percentage Willow Bark,
about 2 weight percentage Myrrh, and
a 20% solution of Arnica Montana, oil in a carrier oil totaling about 0.8 to about 2 weight percentage of the chemical composition,
in from about 92% to about 80% weight percentage of a cream base of equal parts shea and aloe butter.

3. The topical analgesic for sensitive skin, the chemical composition of claim 1 further comprising:
about 4 weight percentage Birch Bark essential oil,
about 2 weight percentage Willow Bark,
about 2 weight percentage Myrrh, and
a 20% solution of Arnica Montana, oil in a carrier oil totaling about 0.8 to about 2 weight percentage of the chemical composition,
in from about 92% to about 80% weight percentage of a cream base of equal parts shea and aloe butter.

4. The topical analgesic for sensitive skin, the chemical composition of claim 1 further comprising:
about 6 weight percentage Birch Bark essential oil,
about 2 weight percentage Myrrh, and
a 20% solution of Arnica Montana, oil in a carrier oil totaling about 0.8 to about 2 weight percentage of the chemical composition,
in from about 92% to about 80% weight percentage of a cream base of equal parts shea and aloe butter.

5. The topical analgesic for sensitive skin, the chemical composition of claim 1 further comprising:
about 4 to about 6 weight percentage Birch Bark essential oil,
about 8 weight percentage Myrrh, and
a 20% solution of Arnica Montana, oil in a carrier oil totaling about 0.8 weight percentage of the chemical composition,
in from about 88% to about 86% weight percentage of a cream base of equal parts shea and aloe butter.

6. The topical analgesic for sensitive skin, the chemical composition of claim 1 further comprising:
about 2 weight percentage Birch Bark essential oil,
about 10 weight percentage Myrrh, and
a 20% solution of Arnica Montana, oil in a carrier oil totaling about 0.8 weight percentage of the chemical composition,
in from about 88% weight percentage of a cream base of equal parts shea and aloe butter.

7. The topical analgesic for sensitive skin, the chemical composition of claim 1 wherein ⅓ by weight of the birch bark is replaced by an equal amount of a compound selected from a group consisting of wintergreen or willow bark, meadowsweet, methyl salicylate, any combination of herbs containing salicin, and combinations thereof.

8. The topical analgesic for sensitive skin, the chemical composition of claim 1 wherein ⅔ by weight of the birch bark is replaced by an equal amount of a compound selected from a group consisting of wintergreen or willow bark, meadowsweet, methyl salicylate, any combination of herbs containing salicin, and combinations thereof.

9. The topical analgesic for sensitive skin, the chemical composition of claim 1 wherein synthetic methyl salicylate is substituted in place of the essential oil of birch bark, wintergreen, or willow bark and in any combination thereof.

10. The topical analgesic for sensitive skin, the chemical composition of claim 1 wherein Grape seed oil or fractionated coconut oil or ben seed oil is used as the base to form a liquid for a roll-on.

11. The topical analgesic for sensitive skin, the chemical composition of claim 1 wherein in place of using essential oils, a ratio of one-ounce raw herbs to six ounces base heated for approximately thirteen hours at 110-120 degrees Fahrenheit.

12. A topical analgesic for sensitive skin, the chemical composition consisting of:
(A) one or more herbs containing the properties of an analgesic such as those selected from a group of herbs containing methyl salicylate, salicin, or menthol, including birch bark, wintergreen, willow bark meadowsweet and peppermint or any other herbs with analgesic properties, such as but not limited to, lavender, everlast Arnica Montana, passion flower, calendula, chamomile, to name just a few;
(B) a transdermal agent, such as Arnica Montana;
(C) one or more herbs containing the properties of an anti-inflammatory, such as but not limited to lavender or birch bark;
(D) Myrrh; and
(E) a base to hold the ingredients together and make it applicable to skin.

13. The topical analgesic for sensitive skin, the chemical composition of claim 12 further consisting of a natural preservative to increase product stability and shelf life of the composition.

14. The topical analgesic for sensitive skin, the chemical composition of claim 12 further consisting of the addition of one or more essential oils for added pain relief using olfactory senses selected from a group consisting of:
about 1-4% weight percentage of Grapefruit essential oil;
about 2-4% weight percentage of Lavender about 2-4% weight percentage of a lavender blend of essential oils;
Allspice, in a concentration of about ½% weight percentage,
neroli and ylang ylang in a concentration of about 1-4% weight percentage;
clove, cinnamon, clary sage in a concentration of about ½% weight percentage, and
vanilla, grapefruit, rose, and geranium in a concentration of about 1-4% weight percentage.

15. The topical analgesic for sensitive skin, as well as certain skin conditions, such as excema and certain types of psoriasis, the chemical composition of claim 12 further consisting of the addition of one or more herbs with the properties of healing cuts or wounds from a group including calendula and/or chaulmoogra.

16. A method of producing a topical analgesic for sensitive skin from essential oils according to the following steps:
heating a base to a liquid state wherein the base is shealoe butter, a 50/50 premix of shea butter and aloe butter is used;
after the butter is melted, supporting carrier oils are added followed by the addition of any desired essential oils.

17. The method of producing a topical analgesic for sensitive skin of claim 16 wherein the supporting carrier oil includes at least one oil selected from a group consisting of:
Calendula in a concentration from about 5% to about 10% weight percentage,
Arnica in a concentration of about 4% weight percentage,
Chaulmoogra in a concentration from about 5% to about 10% weight percentage, and
Aloe Vera, when the base does not already contain aloe, in a concentration of about 10% weight percentage.

18. The method of producing a topical analgesic for sensitive skin of claim 16 wherein
the essential oils contribute to pain relief, at least one oil selected from a group consisting of:
Birch bark in a concentration from about 1-2% weight percentage,
Ginger in a concentration from about ½% to about 1% weight percentage, and
Coriander in a concentration of about 1% weight percentage, and
the essential oils providing scents to the olfactory sense to contribute to pain relief, at least one oil selected from a group consisting of:
Lavender in a concentration from about 3% to about 5% weight percentage,
Myrrh in a concentration from about 8% to about 10% weight percentage, and
Allspice, in a concentration of about ½% weight percentage,
clove, cinnamon, clary sage in a concentration of about ½% weight percentage, and
vanilla, grapefruit, rose, and geranium in a concentration of about 1-4% weight percentage.
